(19) United States
(12) Patent Application Publication
(10) Pub. No.: US 2014/0014114 A1
(43) Pub. Date: Jan. 16, 2014

(54) LARYNGEAL MASK AIRWAY

(71) Applicant: Donald Munro Miller, Cape Town (ZA)

(72) Inventor: Donald Munro Miller, Cape Town (ZA)

(21) Appl. No.: 14/005,956

(22) PCT Filed: Mar. 22, 2012
(86) PCT No.: PCT/IB2012/051364

§ 371 Date: Sep. 18, 2013

(30) Foreign Application Priority Data
Mar. 22, 2011 (GB) 1104835.2

(51) Int. Cl. A61M 16/04

(52) U.S. Cl. CPC A61M 16/0434
USPC 128/207.15

(57) ABSTRACT

An airway device (10) is provided for sealing a patient's pharynx (P), the device comprising an elongate flexible chamber (12) of resilient material with a leading end (14) and a trailing end (16) and being bounded by top (18) and bottom (19) elongate surfaces. The top surface has raised ridge (20) across it to seal at the base (B) of the tongue (T) and the bottom surface is shaped to seal at the back of the patient's throat. An opening (26) is defined in the top surface and serves to place the chamber in communication with the laryngeal inlet (L) of the patient. A gutter (28) extends from the leading edge along the length of the bottom surface and forms a funnel (27) at the trailing end. Instruments passed along the outside of the stem are received in the funnel and directed along the gutter to the oesophagus (O) of the patient. A hollow stem (40) protrudes from the trailing end of the chamber for passing through the patient's mouth and has an attachment formation (42) with a 22 mm diameter, for attaching to external ventilation apparatus.

### FIELD OF THE INVENTION

The present invention relates to respiratory apparatus in the form of an artificial airway device for placement into the oropharynx of an unconscious patient.

### BACKGROUND TO THE INVENTION

In order to support respiration and therefore life, an unconscious patient may require some or all of the following objectives, namely: the maintenance of airway patency, attachment to respiratory apparatus, either spontaneous or controlled positive pressure ventilation, prevention of inhalation into the lungs of extraneous matter such as vomitus or blood and permitting the passage of a tracheal tube into the trachea and/or gastric tube or gastroscope into the oesophagus.

During anaesthesia or resuscitation this may be achieved by means of an endotracheal tube with an inflatable cuff around the end which is placed with the help of other instruments within the trachea, or a laryngeal mask airway (LMA™). The latter may involve the use of an inflatable cuff at the end of a tube, the end of which is placed around the entrance to the larynx and within the pharynx, or an oesophageal obturator airway (EOA) named “Combitube®” or derivative (Suction Laryngeal Tube). This comprises a double lumen double cuffed tube, the longer tube with attached cuff passes into the oesophagus for the purpose of sealing and isolating contents which may enter the oesophagus from below or to prevent the escape of gas under pressure from above from entering the stomach. The shorter tube for ventilating the lungs ends within the pharynx, the oro-nasal outlet from the pharynx being sealed off within the pharynx by means of the second cuff which surrounds both tubes, which, when inflated, allows for positive pressure to develop within the pharynx. Sometimes, an oral or nasal airway tube for preventing obstruction of the airway is used in combination with a facemask.

More recently a cuffed oro-pharyngeal airway (COPA®) has been introduced by Mallinckrodt Medical, Inc, (U.S. Pat. No. 5,743,256) which can be used to achieve some of the objectives stated above but fails to protect the lungs from extraneous matter that enters the pharynx from entering the lungs. It is no longer in production. The glottic aperture seal airway of Augustine Medical Inc. (WO 98/16273) purports to achieve this advantage, however, it is not as reliable as was originally hoped. Numerous other double cuff inflating devices are appearing in the current market and may be classed as derivatives of the EOA and COPA® above, e.g. that of Sato et al (U.S. Pat. No. 5,743,258).

The maximum inflation pressure that can be used before gas leakage occurs around the cuffs limits the application of controlled ventilation by the latter two methods. They also run the risk of inflating the stomach and do not provide a secure airway from the possible aspiration of vomitus. The LMA™ provides a partial seal of the oesophagus but regurgitation, if it gets past the seal, is more likely to pass into the bowl that is surrounded by an inflatable bag cavity in the case of the LMA™ from whence the contents are easily funneled into the larynx. The COPA® and the more recent “Cobra” airways do not provide any seal of the oesophagus. The Combitube would appear to be an effective device, for controlled ventilation, sealing off the oesophagus, but its correct placement can pose problems, either too deep or not deep enough. It is also rather elaborate and expensive. The LMA™ would appear to be ideal except for the fact that the pressures that can be generated in controlled ventilation are limited as the mask could be dislodged at higher inflation pressures. In addition, although it is partially effective in isolating the airway from extraneous matter in the pharynx, should any extraneous matter enter the lumen of the mask, which does not provide a high quality isolation of the trachea from pharyngeal matter, it will tend to be funneled into the larynx. To overcome this disadvantage an improved LMA™ named the ‘ProSeal’ LMA™ incorporates a moderate bore tube for removing liquid that may accumulate in the mask region of the airway by suction or siphonage and is disclosed in JP 2-283378. The placement in the trachea of the endotracheal tube is the most effective means of achieving all of the above objectives, however, its use requires experience, skill and the use of a laryngoscope, which in turn has its own unwanted side effects consequent upon powerful neural reflex actions. Its placement may also require the use of muscle relaxing drugs.

All the above devices are made of soft, flexible materials and for a removably secure connection to respiratory apparatus necessitate a connecting mechanism with sufficient tenacity to prevent unintentional disconnection. The force required for detachment of the respiratory apparatus from the airway device should exceed 30 Newtons. For this to be achieved usually requires a hard plastic male connector that is applied (via barbed fitting or glued or stretched with good quality friction fit) to the softer material of the airway device. On occasions this junction has come adrift and constitutes a potential hazard which would be beneficial to avoid, if possible.

Another complication of using supraglottic airways is the incidence of damage to certain nerves, namely the hypoglossal and recurrent laryngeal nerves due to pressure effects of cuff inflating airway devices in the pharynx.

A recent innovation by the inventor of the present invention, relates to a combined obturator and airway device named the SLIPA™ (WO 02/32490). This device addresses many of the objectives named above, for supporting an unconscious patient, but like all current devices, it uses a standard 15 mm attachment connector that conforms to BS 5356 standard for conical connectors. These attachments are made of different material from the airway devices, with the result that there is occasionally insufficient frictional grip between the connectors and the devices and the connectors can therefore potentially come adrift, which highlights a potential risk factor. This is likely to be an even bigger problem should smaller sizes of the device need to be made for children. In addition, these connectors restrict the diameter of instruments that may pass through them.

If gases are to be administered to a patient via an airway device, such as the SLIPA™ device, this typically has to be done in the apparatus that is connected to the standard 15 mm connector. However, there is considerable dead space between the point of introduction of the gases and the patient's lungs and it would be preferable if the gases could be introduced closer to the patent's lungs.

Further, the SLIPA™ device does theoretically permit the passage of a tracheal tube into the trachea, but this has to be achieved with considerable difficulty through an aperture on top of the device, intended to be in communication with the patient's pharynx and the geometry of the device makes it almost impossible to pass a tracheal tube through this aperture. Further, this device does not permit the passage of a gastric tube into the oesophagus, at all.

The SLIPA™ device, unlike many other airway devices, is pre-shaped with an indentation just below the sealing ridge, which may be partially effective in preventing nerve injury at a particular site. However, it may still be possible for it to exert pressure on the nerve near the tips of the patient's hyoid bones and/or the hypoglossal nerve, causing nerve dysfunction or discomfort or pain to the patient.

The present invention seeks to address a number of apparently contradictory objectives, which fall into three groups.

The first group of objectives seek to provide an airway device that is good for the management of the difficult airway and the first requirement for this is for blind intubation, as can be achieved with the intubating laryngeal mask (iLMA), the LMA classic and the i-gel (which are not nearly as effective but can also be used for the same purpose) and the AirQ device by Daniel Cook (which is almost as effective). The second requirement is to be able to use the airway device with curved optical stylets so that visualization of the vocal cords may be achieved quickly and simply in case the blind intubation technique fails. The third requirement is to have a device that is easy and safe to use without having connectors obstructing intubation objectives and also without having to struggle (fiddle) with having to remove connectors during complicated airway management procedures. The iLMA, LMA classic and i-gel devices cannot be used with curved optical stylets because of the design of the 15 mm connectors that do not allow this. The AirQ device is specifically designed so that its 15 mm connector can be easily removed and reinserted and this allows for instruments such as intubation objectives to be used with the connector removed. A forth requirement is to limit the risk that parts of the respiratory apparatus may become disconnected during use.

The second group of objectives (without any sacrifice of the first group of objectives, above) seek to provide an airway device that has the simplicity of the SLIPA airway (WO 02/32490), that is not suitable for blind intubation, and yet retains its advantages of an effective capacity storage mechanism for aspiration protection and a device that can be used in children. None of the above airway devices allow for a significant storage mechanism for aspiration protection. The i-gel device does have a small gastric tube channel but its design limits the size of the gastric tube to less than 5 mm diameter and its aspiration protection characteristics are limited by the limited effectiveness non-inflatable oesophageal seal. Gastric tubes in other devices interfere seriously with the intubation characteristics of the device such as the non-midline airway tube in the ProSeal and LMA supreme devices.

The manufacture of the SLIPA airway cannot be achieved in children because the simple manufacturing blow moulding technique necessitates a connector to fit tightly on the outside of the stem of the device if it is not to leak at the connector site. The wall thickness of a blow moulded device for children is too thick and the device is too hard so a paediatric size SLIPA is not possible.

The third group of objectives seek to access the oesophagus with large bore gastric instruments such as gastric tubes, gastroscopes and transesophageal echo devices, in both children and adults. None of the present airway devices are suitable in both adults and children.

### BRIEF DESCRIPTION OF THE INVENTION

According to a first aspect of the present invention there is provided an airway device for sealing an unconscious patient's pharynx without penetration of the device into the larynx, i.e. a supralaryngeal airway, the device comprising:
a hollow stem, configured to be in communication with the patient's laryngeal inlet and for extending through the mouth of the patient, to external respiratory apparatus, e.g. ventilation apparatus; and
a sealing component, configured to seal against the walls of the patient's pharynx;
   wherein the device includes an attachment formation at the end of the stem that is of an appropriate diameter to be connected by means of removably secure friction attachment, to the firm outside wall of the external respiratory apparatus, said attachment formation having a nominal diameter of about 22 mm.

The attachment formation and the stem may be continuous, may be of the same material, and may form a unitary article. The attachment formation may be a tapered female formation and may comply with ISO 5356 conical connector specifications.

The sealing component may comprise:
a body of resilient material defining an elongate flexible saccular chamber, shaped to seal against the walls of the patient's pharynx, the body having a leading end and a trailing end and being bounded by first and second opposed surfaces, the first surface defining a protuberance that is shaped to conform with the shape of the base of the tongue to seal against the base of the tongue and the second surface being shaped to seal against the back of the patient's throat;
an opening defined in the first surface of the body, the opening being on the same side of the protuberance as the leading end and serving to place the inside of the chamber in communication with the laryngeal inlet of the patient; and
wherein the hollow stem protrudes from the chamber at the trailing end of the chamber for connecting the inside of the chamber, through the mouth of the patient, to the external respiratory apparatus.

The long axis of the stem may be oriented at an angle of between 45 degrees and 120 degrees with respect to a longitudinal axis of the body.

The body may further include a gutter, defined in the second surface, the gutter extending from the trailing edge to the leading edge, providing a means through which a tube may be inserted into the oesophagus of the patient by passing along the length of the gutter between the second surface and the posterior pharyngeal wall. The gutter may form a funnel at the trailing end of the chamber that is wider at the trailing end of the chamber, than the width of the stem where it is connected to the chamber.

According to another aspect of the present invention there is provided an airway device for sealing an unconscious patient's pharynx without penetration of the device into the larynx, the device comprising:
a body of resilient material defining an elongate flexible saccular chamber, shaped to seal against the walls of the patient's pharynx, the body having a leading end and a trailing end and being bounded by first and second opposed surfaces, the first surface defining a protuberance that is shaped to conform with the shape of the base of the tongue to seal against the base of the tongue and the second surface being shaped to seal against the back of the patient's throat;
an opening defined in the first surface of the body, the opening being on the same side of the protuberance as the leading end and serving to place the inside of the chamber in communication with the laryngeal inlet of the patient; and
a hollow stem protruding from the chamber at the trailing end of the chamber for connecting the inside of the chamber, through the mouth of the patient, to external respiratory apparatus;
wherein the body further includes a gutter in the second surface extending from the trailing end to the leading end of the chamber, said gutter forming a funnel and the trailing end of the chamber and said funnel being wider at the trailing end of the chamber, than the width of the stem where it is connected to the chamber, the funnel and gutter providing a means through which a tube may be inserted into the oesophagus of the patient by passing along the length of the gutter between the second surface and the posterior pharyngeal wall

The trailing edge dimensions of the gutter in the second surface are greater than its dimensions at the leading edge, thus giving the gutter a trumpet shape in the second surface, extending in a direction from the leading end of the body along the entire length of the second surface.

The stem of the device may comprise a hollow central channel and may be shaped to provide at least one lateral gutter on one or both sides of the central channel with a concave surface on the same side as the first surface of the chamber, the gutter or gutters extending from below the attachment formation for most of the length of the stem and ending before the chamber section, where the curvature of the stem bends in the direction of the first surface of the chamber and where the stem has its smallest radius of curvature.

The funnel in the second surface is wide enough to be alligned with the lateral gutter or gutters of the stem and serving to provide an entrance means through which a tube or flexible scope can be inserted into the oesophagus of the patient that may be passed along the lateral gutter or gutters and directed centrally as it passes through the funnel behind the second surface at the trailing edge of the chamber gutter.

The opening defined in the first surface may be located at the trailing end of an elongate recess defined in the first surface and extending in a direction from the leading end of the body for part of the length of the first surface, to provide a flow path for gases from the opening in the first surface to the patient's laryngeal opening, even in the event that the patient has a long epiglottis overlaying the opening.

The body may define indentations at lateral locations on the first surface, on the same side of the protuberance as the leading end, said indentations corresponding generally to the location of the tips of the patient's hyoid bones, to relieve pressure over the tips of these bones and the associated hypoglossal nerve that is commonly located near this site.

The leading end of the body may be slanted with a leading edge of the second surface extending further than a leading edge of the first surface, in the direction of the leading end of the body, so that the leading end of the body will slide off the posterior pharyngeal wall at the base of the tongue during insertion or placement of the device in the patient's pharynx.

The first surface may be generally concave along its length, in order to conform with the shape of the patient's pharynx, said concavity extending up to a leading edge of the first surface for sealing abutment against the anterior oesophageal wall and firmer posterior tracheal wall, so that on insertion of the body into the patient's pharynx, the leading edge will not catch on the epiglottis at the base of the tongue and so that it will seal into the crescent shaped entrance into the oesophagus.

The protuberance on the first surface may be a crescent shaped raised ridge extending transversely across the first surface and the ridge may have two lateral lumps that are shaped to conform to the shape of the patient's glosso-epiglottic fold at the base of the tongue.

The device may include an attachment formation at the end of the stem that is to be connected to the external respiratory apparatus, the attachment formation having a nominal diameter of about 22 mm.

The diameter of the attachment formation may be larger than the diameter of the stem, the attachment formation and the stem may be continuous and may form a unitary article, made of the same material, preferably a material characterised by flexibility and resilience. The attachment formation may preferably be a tapered female formation and may comply with ISO 5356 conical connector specifications. More specifically, the attachment formation may preferably be a tapered female formation that will be stretchable over an ISO 5356 male conical connector so that the detachment force needed to disconnect the attachment formation from the connector, will exceed the required standard of 30 Newtons.

### BRIEF DESCRIPTION OF THE DRAWINGS

For a better understanding of the present invention and to show how it may be carried into effect, the invention will now be described by way of non-limiting example with reference to the accompanying drawings, in which:

FIG. 1 is a side view of an airway device in accordance with the present invention;

FIG. 2 is a view from the trailing edge aspect of the device of FIG. 1;

FIG. 3 is a three dimensional inferior (bottom) diagonal view of the device of FIG. 1;

FIG. 4 is a superior (top) view of the device of FIG. 1, showing an upper or first surface of a chamber of the device;

FIG. 5 is a diagonal superior view of the device of FIG. 1, showing the first or upper surface of the chamber;

FIG. 6 is an inferior view of the device of FIG. 1, showing a second or inferior surface;

FIG. 7 is a sectional side view of the device of FIG. 1, in use in the pharynx of a patient;

FIG. 8 is a sectional side view of the device of FIG. 1, in use in the pharynx of a patient, showing a line of passage of a gastroscope or gastric tube to the eosophagus;

FIG. 9a is a superior view of the first surface of the device of FIG. 1; and with

FIG. 9b is an inferior view of the second surface of the device of FIG. 1, showing the passage of a gastroscope or gastric tube from a patient's mouth to oesophagus, with a dotted line representing its passage behind the surfaces of the airway device.

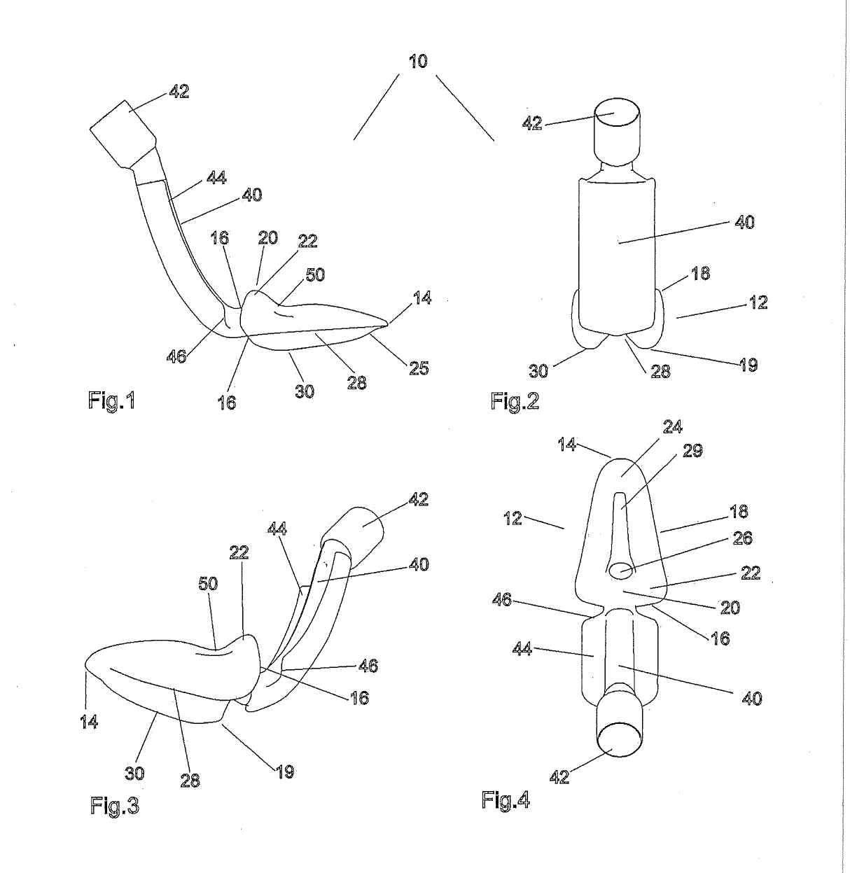

### DETAILED DESCRIPTION OF THE DRAWINGS

Referring to the drawings, an airway device in accordance with the present invention is generally indicated by reference numeral 10.

The device 10 is intended to be inserted or placed inside the pharynx of an unconscious patient during anaesthesia or resuscitation, to maintain patency of the airway by sealing the patient's pharynx P without penetrating into the larynx L, among a number of other functions. One of the functions of the device 10 is to obstruct the oesophagus O to prevent gas passing into the oesophagus and regurgitated fluid flowing from the oesophagus into the airway.

The device 10 includes a sealing component in the form of a body of resilient material such as a polymeric material, in the form of an elongate flexible saccular chamber 12, the body having a leading end 14 and a trailing end 16 and being bounded by first 18 and second 19 opposed elongate surfaces. The orientation of the body may vary, but it is intended for use on a patient in a supine position (face upwards), with the first surface 18 facing upwardly and the second surface 19 facing downwardly and with the leading end 14 received in the opening of the oesophagus O. Accordingly, for the sake of clarity, the first surface will be referred to as the top surface 18 and the second surface will be referred to as the bottom surface 19. The description of the views illustrated in the drawings, are also based on the orientation of the device, when in use.

Some aspects of the present invention are also applicable to airway devices with other sealing components, such as inflatable cuffs.

The chamber 12 is shaped to seal against the walls of the patient's pharynx P and the top surface 18 has a protuberance in the form of a raised ridge 20 extending transversely across it, that is shaped to conform with the shape of the base B of the patient's tongue T, so that it can seal against the base of the tongue, in the vicinity of the glosso-epiglottic fold Ge. In the illustrated embodiments, the ridge 20 has two lateral lumps 22 that stand proud of the ridge extending between them and that are shaped to conform to the shape of the lateral aspects of the glosso-epiglottic fold Ge, to enhance the sealing function of the chamber 12 and to assist in keeping the device 10 in position.

The device includes a hollow stem 40 that protrudes from the trailing end 16 of the chamber 12 and that has a curved shape to extend around the patient's tongue T, through the oral cavity and mouth M to be connected to external respiratory apparatus, typically ventilating apparatus. The stem 40 thus connects the inside of the chamber 12 to the external respiratory apparatus. In order for the stem 40 to extend reasonably comfortably from the chamber 12 to the outside of the patient's mouth, it has to have a general orientation of about 45 degrees to 120 degrees with respect to a longitudinal axis in the direction of elongation of the chamber. The relative stiffness of the stem 40 compared to that of the chamber 12 may cause the chamber to buckle resiliently at the junction of the stem and chamber when the axis of the stem becomes perpendicular with respect to the longitudinal axis of the chamber, as is typically required when in position after insertion or placement of the device 10.

The top surface 18 is generally concavely shaped along its length, in order to conform to the shape of the sealing sites of the device in the patient's pharynx P and the concavity extends up to the leading edge 24 of the top surface. This helps to prevent the chamber and particularly the leading edge, from catching on the epiglottis Ep on insertion or placement of the device 10 into the patient's pharynx P. The concavity of the leading edge 24 gives it a crescent shape, which further helps to seal the leading end 14 against the crescent shaped entrance into the oesophagus O.

The leading end 14 of the chamber 12 is slanted (as can best be seen in FIG. 1) with the leading edge 25 of the bottom surface 19 extending beyond the leading edge 24 of the top surface 18, in the direction of the leading end, i.e. upwardly and forwardly. The slanted shape of the leading end 14 causes it to slide off the back of the pharyngeal wall at the base of the tongue and the concavity of the leading edge surface 24 enables the device to slide off the back of the epiglottis Ep during insertion or placement of the device 10 in the patient's pharynx P.

An elongate, recess or deep top groove 29 is defined in the top surface 18, extending longitudinally in a direction from the vicinity of the leading end (14), preferably from a little way short of the leading end, for part of the length of the top surface and stopping short of the ridge 20 and an opening 26 that is defined in the top surface, recessed within the top groove, generally at the end of the groove closest to the ridge, i.e. at the trailing end of the groove. The main opening 26 is thus on the same side of the ridge 20 as the leading end 14 and can serve to place the inside of the chamber 12 in communication with the laryngeal inlet L of the patient.

The groove 29 helps to provide alternative flow passages for gases between the inside of the chamber 12 and the patient's laryngeal opening L, e.g. the groove can help to maintain a passage extending from the main opening 26 In the event of a long epiglottis Ep overlaying the main opening 26, the groove 29 provides a passage for gas to flow between the chamber 12 and the larynx L, even if the epiglottis Ep blocks the main opening.

Further, the opening 26, along with the top groove 29 and concave top surface 18 are configured such that in use, they collect liquid and drain it under gravity into the inside of the chamber 12, where it collects under gravity within the chamber 12, well out of the way of the opening 26 through which gases are intended to flow under normal circumstances. This is necessary to prevent liquid from entering the patient's larynx L, trachea Tr or lungs. The liquid collected in the chamber 12 can be removed from the chamber, as required, by aspirating it through a catheter inserted into the chamber via the stem 40.

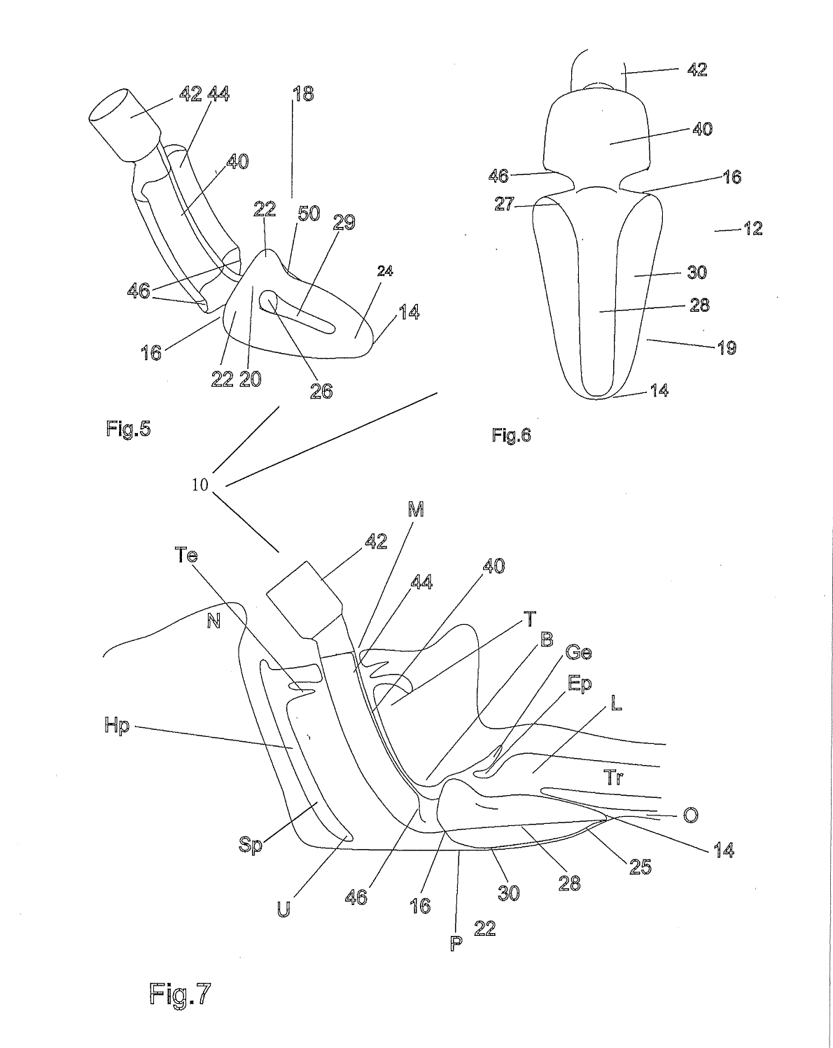

The bottom surface 19 is shaped along its length to conform to the shape of the back of the patient's throat, to seal against the posterior lateral pharyngeal walls P (as shown in FIG. 7) at this site.

An elongate gutter 28 is defined in the bottom surface 19, extending longitudinally in a direction from the leading end 14 for the length of the bottom surface to the trailing end 16 of the chamber 12. As can be seen in FIG. 6, the gutter 28 widens in the region of the trailing end 16 to form a trumpet-shaped funnel 27 with its widest dimension at the trailing end 16, where it is wider than the central part of the stem 40 (i.e. the stem excluding the wings mentioned below) and is wider than the stem at the point where it is connected to the chamber 12. Two elongate protuberances 30 extend laterally alongside the gutter 28 in the bottom surface.

As can be seen in FIGS. 1,3 and 8, there are indentations 50 on the top surface 18 on the leading edge side of the ridge 20 and next to the lateral lumps 22. The indentations are in the directions both towards the bottom surface 19 and inwardly towards the centre of the chamber 12 to make a space for the purpose of relieving pressure, when in use, over the tip of the hyoid bone and the closely associated hypoglossal nerve that may normally be located near this site.

At the end of the stem 40 that is intended to be connected to external respiratory apparatus, it has an attachment formation in the form of a non-standard yet ISO 5356 compliant 22 mm female tapered attachment formation 42. The stem 40 itself may be much narrower than the attachment formation 42, with a typical nominal internal diameter of about 14 mm, as opposed to the 22 mm nominal diameter of the attachment formation. However, the stem 40 includes lateral wing formations that each define a gutter 44 on its front and as a result, the stem is much wider than the attachment formation 42 in the lateral direction. Each gutter 44 ends at a shoulder formation 46 at its lower end, at a point where the stem 40 has its smallest radius of curvature and starts to curve towards the top surface 18. The reason for the narrower stem 40 is that it would be difficult to insert a device with a stem that had a larger forward and backward dimension and/or would be uncomfortable to the patient. A small diameter dimension stem 40 is present at the junction between the stem and chamber 12 in order to improve flexibility of the junction between the stem 40 and the chamber 12.

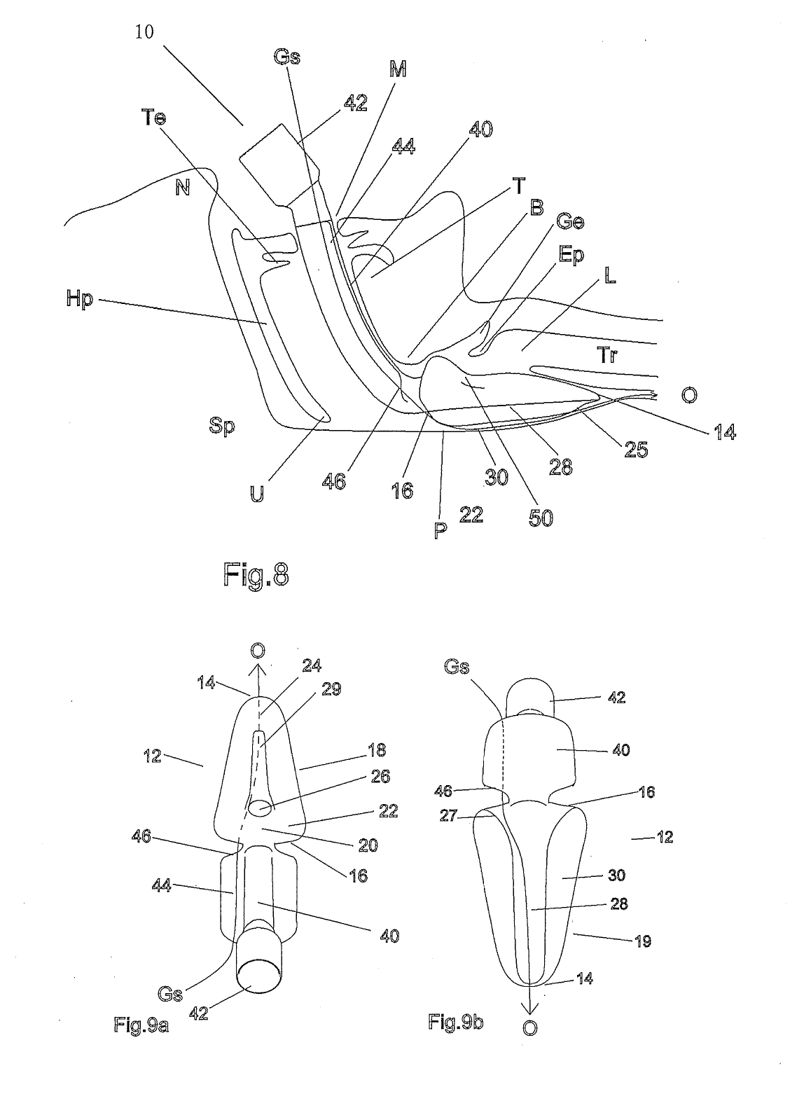

As can be seen in FIGS. 8, 9a and 9b, the geometries of the gutters 44 and bottom gutter 28 allow the passage of a narrow or wide bore gastric tube or gastroscope Gs via the bottom gutter into the oesophagus O and stomach to remove liquids or view the stomach, by the passing of an appropriate diameter and length tube or scope via one of the gutters 44 in the front of the stem 40 then directed between the chamber 12 and the posterior pharyngeal wall P, along the gutter 28 from the trailing end 16 to the leading end 14 towards the oesophagus. The funnel 27 is wide enough to receive the gastric tube or gastroscope Gs that has passed along a lateral gutter 44 and laterally past the stem 40 at its connection to the chamber, and the funnel directs the gastric tube or gastroscope Gs to travel centrally along the gutter 28 towards the oesophagus O. The path for passage of the gastric tube or gastroscope Gs is shown in FIGS. 8, 9a and 9b with arrow heads pointing in the direction of the oesophagus O.

It is also possible to pass a wider bore tracheal tube through the device 10 by feeding it through the attachment formation 42, 40, chamber 12 and opening 26, into the trachea Tr, while the patient's head is in the neutral position.

The use of a much wider attachment formation 42 in the present invention than other supraglottic airways provides for a wider entrance to the airway device 10 and thus for the passage of other devices such as tracheal tubes and other instruments such as curved optical stylets, which may be needed to pass through the attachment formation under circumstances of difficult airway management.

As mentioned above, existing airway devices are typically made of soft, flexible materials and in order to allow them to be attached to standard ISO compliant fittings, they are provided with standard 15 mm male attachment connectors that are made of different, harder material from the airway devices. The attachment formation 42 and the stem 40 are continuous and form a unitary article and since they do not need to be attached together, e.g. by barbed fittings, adhesive, frictional attachment, etc they can be made of a non-rigid material. This is also advantageous since the attachment formation 42 is a female attachment formation that will be pressed onto a male fitting, in use, and its resilient flexibility will allow it to expand to some extent while being attached and to press against the male fitting, thus enhancing its seal and firmness of its frictional attachment. The entire device 10, including the chamber 12, stem 40 and attachment formation 42 is typically a unitary moulding or single component made from the same resiliently flexible material. With the whole device 10 comprising one component with the attachment formation integrally connected to the stem 40, there is practically no risk that the attachment formation may come adrift of the stem.

A particular advantage of the nominal 22 mm diameter female connector design is that it makes it possible to provide an airway device 10 that is suitable for children. In order to blow mould a thinner walled device 10 with softer walls that is suitable for children, it is preferable to use an attachment arrangement that is a unitary device of the same material. So the safest means of achieving this is by means of a 22 female connector arrangement that allows for firm attachment with thinner walls.

### KEY TO ANATOMICAL REFERENCES IN FIGS. 7 & 8

B Base of tongue

Ep Epiglottis

Ge Glosso-epiglottic fold

Hp Hard palate

L Laryngeal inlet

M Mouth

N Nasopharynx

O Oesophagus

P Pharynx

Sp Soft palate

Te Teeth

T Tongue

Tr Trachea

U Uvula

1-30. (canceled)

31. An airway device for sealing a patient's pharynx without penetration of the device into the larynx, the device comprising:
a hollow stem, configured to be in communication with the patient's laryngeal inlet and for extending through the mouth of the patient, to external respiratory apparatus;
a sealing component, configured to seal against the walls of the patient's pharynx; and
an attachment formation at the end of the stem that is of an appropriate diameter to be connected by means of removably secure friction attachment to the external respiratory apparatus, said attachment formation having a nominal diameter of about 22 mm.

32. A device as claimed in claim 31, wherein the attachment formation and the stem are continuous and form a unitary article.

33. A device as claimed in claim 32, wherein the attachment formation and the stem are made of the same material.

34. A device as claimed in claim 31, wherein the attachment formation is a tapered female formation.

35. A device as claimed in claim 31, wherein the attachment formation complies with ISO 5356 conical connector specifications.

36. A device as claimed in claim 31, wherein the sealing component comprises:
a body of resilient material defining an elongate flexible saccular chamber, shaped to seal against the walls of the patient's pharynx, the body having a leading end and a trailing end and being bounded by first and second opposed surfaces, the first surface defining a protuberance that is shaped to conform with the shape of the base of the tongue to seal against the base of the tongue and the second surface being shaped to seal against the back of the patient's throat;
an opening defined in the first surface of the body, the opening being on the same side of the protuberance as the leading end and serving to place the inside of the chamber in communication with the laryngeal inlet of the patient; and
wherein the hollow stem protrudes from the chamber at the trailing end of the chamber for connecting the inside of the chamber, through the mouth of the patient, to the external respiratory apparatus.

37. A device as claimed in claim 36, wherein the body includes a gutter in the second surface, that extends from the trailing end to the leading end of the chamber.

38. A device as claimed in claim 37, wherein the gutter forms a funnel at the trailing end of the chamber, said funnel being wider at the trailing end of the chamber than the width of the stem where it is connected to the chamber.

39. A device as claimed in claim 31, wherein the stem is shaped to define at least one lateral gutter, said gutter extending from below the attachment formation and ending short of a point where the stem curves towards the first surface.

40. A device as claimed in claim 36, wherein the body includes a gutter in the second surface, said gutter extending from the trailing end to the leading end of the chamber and said gutter forming a funnel at the trailing end, said funnel being wider than the width of the stem where it is connected to the chamber, wherein the stem is shaped to define at least one lateral gutter, said funnel being aligned with said lateral gutter.

41. A device as claimed in claim 36, wherein the opening defined in the first surface is disposed in an elongate recess defined in the first surface and extending in a direction from the vicinity of the leading end of the body for part of the length of the first surface, the opening being defined at the trailing end of the recess.

42. A device as claimed in claim 36, wherein the body defines indentations at lateral locations on the first surface, on the same side of the protuberance as the leading end, said indentations corresponding generally to the locations of the tips of the patient's hyoid bones.

43. A device as claimed in claim 36, wherein the leading end of the body is slanted with a leading edge of the second surface extending further than a leading edge of the first surface, in the direction of the leading end of the body.

44. A device as claimed in claim 36, wherein the first surface is generally concave along its length, said concavity extending up to a leading edge of the first surface.

45. A device as claimed in claim 36, wherein the protuberance is a raised ridge.

46. A device as claimed in claim 45, wherein the ridge is crescent shaped and has two lateral lumps that are shaped to conform, at least in part, to the shape of the patient's glosso-epiglottic fold at the base of the tongue.

47. An airway device for sealing a patient's pharynx without penetration of the device into the larynx, the device comprising:
a body of resilient material defining an elongate flexible saccular chamber, shaped to seal against the walls of the patient's pharynx, the body having a leading end and a trailing end and being bounded by first and second opposed surfaces, the first surface defining a protuberance that is shaped to conform with the shape of the base of the tongue to seal against the base of the tongue and the second surface being shaped to seal against the back of the patient's throat;
an opening defined in the first surface of the body, the opening being on the same side of the protuberance as the leading end and serving to place the inside of the chamber in communication with the laryngeal inlet of the patient; and
a hollow stem protruding from the chamber at the trailing end of the chamber for connecting the inside of the chamber, through the mouth of the patient, to external respiratory apparatus,
wherein the body includes a gutter in the second surface, that extends from the trailing end to the leading end of the chamber, said gutter forming a funnel at the trailing end of the chamber, said funnel being wider at the trailing end of the chamber, than the width of the stem where it is connected to the chamber.

48. A device as claimed in claim 47, wherein the stem is shaped to define at least one lateral gutter, said gutter extending from below the attachment formation and ending short from a point where the stem has its smallest radius of curvature.

49. A device as claimed in claim 47, wherein the stem is shaped to define at least one lateral gutter, said funnel being aligned with said lateral gutter.

50. A device as claimed in claim 47, wherein the opening defined in the first surface is disposed in an elongate recess defined in the first surface and extending in a direction from the vicinity of the leading end of the body for part of the length of the first surface, the opening being defined at the trailing end of the recess.

51. A device as claimed in claim 47, wherein the body defines indentations at lateral locations on the first surface, on the same side of the protuberance as the leading end, said indentations corresponding generally to the locations of the tips of the patient's hyoid bones.

52. A device as claimed in claim 47, wherein the leading end of the body is slanted with a leading edge of the second surface extending further that a leading edge of the first surface, in the direction of the leading end of the body.

53. A device as claimed in claim 47, wherein the first surface is generally concave along its length, said concavity extending up to a leading edge of the first surface.

54. A device as claimed in claim 47, wherein the protuberance is a raised ridge.

55. A device as claimed in claim 54, wherein the ridge is crescent shaped and has two lateral lumps that are shaped to conform, at least in part, to the shape of the patient's glosso-epiglottic fold at the base of the tongue.

56. A device as claimed in claim 47, wherein the device includes an attachment formation at the end of the stem that is to be connected to the external respiratory apparatus, said attachment formation having a nominal diameter of about 22 mm.

57. A device as claimed in claim 56, wherein the attachment formation and the stem are continuous and form a unitary article.

58. A device as claimed in claim 57, wherein the attachment formation and the stem are made of the same material.

59. A device as claimed in claim 56, wherein the attachment formation is a tapered female formation.

60. A device as claimed in claim 56, wherein the attachment formation complies with ISO 5356 conical connector specifications.
